SEQUENCE LISTING (1 sequence)

SEQ ID NO: 1 - DNA, 1611 bp, Colletotrichum siamese
atgggaacttcgccgaccgacgcctcgaccgggggtgacaccaaatcgcccaaacagtcaaccaatgccactcctcctcgtcaagacactgctcccgaagtaaagcaacctgacccgagtttgaagcccgatgccgacggcgcgaaacctcttggacctcccccacgacccggacaagctacaggaaacactcccgattactttggcggcgtagcagctgcatccctcagtctggagcccaatcccttcgagcagtctttcggcggcggagcgccagagacacccggtggcacgaaattgccatctgtagccgctttgacatctccatcctcgctgcttcctggcagcggtgctacaccgttcaactggggaggaggctcgcttcgaacgggccccctgagtccagctatgctgtccggccctacaagtgactacttcagcgatacgcaccatctccgaggcgggttccctacaccgaacgagtcttcattgaggacgggcttgacacccggaggcagtggatctatgttcccagcccctagtcccaactctcagcaactgttcgcccagcttgccagtggtggcgctacgcccagtacgattgacttccatcgtacggctttgagtgctgcggcgaagcgtgaaacccagaaccagaaccaaaaccagcctcagaaccagcagcaacagcctgcttcttcggtcacttctcagccgcaggagatggccaacggtgtcgcagttgtcaaaaccgaagcaaagcagcctggtgccttcgacccccacgataacgacgcggcgaacggtctgttcatgcttgctcagggtagaaacggagcgcagccaccgggatacgcccccgcgacgcaggctcagtcacatccgcaacctccgccagcgcccgcccaaagtgtcgagacttctccccaaatgggcagcgtaaacggtgcaggatctgccggtgctggatcttctgtaagaggcgtaagcgaaggtggaagcgccgcctcggacgagagcgagcaggctcgaccttctacccgtggcaagggcaagcgtaattcgaccggtggcgcggtcaccaacggccgcaggaaggctgaagaacctccagcaaaggcacccccaagcaagaaggccaagaccaatctgtcgcctcctcctgacatgaatggagatgaaagccactcagacgacgacgatgatatgaagaagcacgacgacaaggaaggcggctccaagtcaaagatgacagatgaggagaagcggaagaacttcttggagcgtaacagagtcgccgccttgaagtgtcgccagcgcaagaagcagtggctggctaatctccagtccaaggttgagctgttcagcagcgagaatgatgcgttgacagcacaaataacgcagctccgcgaggaagttgtcaacctgaagacgctacttttggcacacaaggactgtcctgtcacccagcaacagggcctccacggcgcgttcatgcaacaggccatggagcccttcaaccctcagatgaacccatacggcatgggtgcgcccatcccccagcaacaagttttggcagctggacagggcgtccagcgacgtttctcatga

(19) United States
(12) Patent Application Publication
(10) Pub. No.: US 2024/0191244 A1
(43) Pub. Date: Jun. 13, 2024

(54) AN ANTHRACIS TRANSCRIPTION FACTOR CsATF1 AND THE APPLICATION THEREOF

(71) Applicant: HAINAN UNIVERSITY, Haikou, Hainan (CN)

(72) Inventors: Chunhua Lin, Haikou, Hainan (CN); Siqi Fang, Haikou, Hainan (CN); Miao Song, Haikou, Hainan (CN); Weiguo Miao, Haikou, Hainan (CN); Wenbo Liu, Haikou, Hainan (CN); Xiao Li, Haikou, Hainan (CN)

(21) Appl. No.: 17/310,660

(22) PCT Filed: May 24, 2021
(86) PCT No.: PCT/CN2021/095554

§ 371 Date: Aug. 16, 2021

(30) Foreign Application Priority Data
May 25, 2020 (CN) 202010447861.X

(51) Int. Cl. C12N 15/80; A01N 63/50; A01N 63/60; C07K 14/37

(52) U.S. Cl. CPC C12N 15/80; A01N 63/50; A01N 63/60; C07K 14/37

(57) ABSTRACT

The present invention provides a colletotrichum transcription factor CsATF1 and its application thereof; the gene which contains 2 introns, encodes 536 amino acids and contains 3 Aft1 domains and 1 BRLZ (basic leucine zipper) domain is an ATF transcription factor in a bZIP transcription factor family. The results of experiments show that the transcription factor is involved in regulating sensitivity of fungi to pyrrole agents such as fludioxonil, indicating that CsATF1 can be applied in preparing a bactericidal enhancer.

### TECHNICAL FIELD OF THE INVENTION

The present invention relates to the field of biotechnology, in particular to a colletotrichum transcription factor CsATF1 and its application thereof.

### BACKGROUND OF THE INVENTION

(1) Transcription factors can inhibit or activate the transcription of some genes and regulate the physiological metabolism, growth and development of organisms.

Transcription factors (TFs) play a critical role in almost all biological processes. Transcription factors, also known as trans-acting factors, refer to DNA binding proteins that can specifically interact with cis-acting elements in a gene promoter region, activate or inhibit transcription of some genes through interactions between the DNA binding proteins and interactions between other associated proteins, thus regulating expression of target genes at a specific intensity in a specific time and space, and regulating physiological metabolism, growth and development of organisms (Latchman, 1997). There are many kinds of transcription factors and Basic leycine zipper (bZip) transcription factors are of a most widely distributed and conserved type in eukaryotic transcription factors. This type of transcription factor has a leucine residue at every 6 amino acids on a peptide chain of protein, is arranged in the same direction of α-helix in spatial conformation and polymerized into dimers by a hydrophobic bonds. The bZIP transcription factors are involved in eukaryotic morphogenesis, disease resistance, seed formation, plant senescence, flower development, biological and abiotic stress response and the like.

(2) The transcription factor CsATF1 is an important component of the bZip transcription factor and a downstream regulatory factor of HOG MAPK.

ATF transcription factors are ATF/CREB family proteins possessed by all eukaryotes. Such genes are mainly involved in regulating oxidative stress response, toxin production and pathogenicity and the like. ATFB genes in Aspergillus parasiticus are not only involved in oxidative stress, but also affect the production of aflatoxin (Wee et al., 2017). Moatfl (Guo et al., 2010) in magnaporthe grisea and Foatf1 of Fusarium oxysporum are involved in regulating oxidative stress response and pathogenicity, and regulating expressions of peroxidase involved in a response to defense response processes of plants. This confirms that a transcription factor VdAtf1 in Verticillium dahliae regulates virulence by regulating nitrogen metabolism. It has been confirmed that the ATF/CREB family proteins are downstream transcription factors of a MAPK (Mitogen-Activated Protein kinases) signaling pathway, which are jointly involved in regulating a variety of stress responses (Zhou et al., 2002). An atfA gene in aspergillus is a downstream transcription gene, i.e. SskA-HogA-AtfA, of a HOG MAPK (High-Osmolarity Glycerol Mitogen-Activated Protein kinases) pathway, and this pathway is involved in regulating sexual reproduction, response to various stresses such as oxidative stress (Hagiwara et al., 2014; Lara-Rojas et al., 2011). There are still limited studies on associated ATF transcription factors in hevea brasiliensis colletotrichum.

### SUMMARY OF THE INVENTION

In view of the disadvantages of the prior art, the present invention provides a Colletotrichum transcription factor CsATF1and its application thereof.

The solution of the present invention includes the following aspects:

A colletotrichum transcription factor CsATF1, wherein the said promoter contains the nucleotide sequence shown in SEQ ID NO: 1.

A protein encoded by the colletotrichum transcription factor CsATF1, wherein the said protein contains the amino acid sequence shown in SEQ ID NO: 2.

A CsATF1 gene knockout vector, comprising following preparation steps: designing primer pairs CsATF1-UP-F/CsATF1-UP-R and CsATF1-D-F/CsATF1-D-R at upstream and downstream sequences of a CsATF1gene encoded reading frame, obtaining an upper arm sequence of the CsATF1 gene and a lower arm sequence behind a C terminal by PCR amplification, and linking the upper arm sequence and the lower arm sequence into a vector pCX62-S, by a method of homologous recombination, to obtain the knockout vector; the nucleotide sequence of the CsATF1 gene is shown in SEQ ID NO:1;
the sequence of primer CsATF1-UP-F is: 5′-GTACCGGGCCCCCCCAGCTGAAGCAGGAGCAACATGGAA-3′
the sequence of primer CSATF1-UP-R is: 5′-CGATACCGTCGACCTCGAGATGACGACGATGATGTATT-3′
the sequence of primer CsATF1-D-F is: 5′-GCTCTCACCGCGGATCCGAGAAGTGATGCGTAATCTG-3′
the sequence of primer CsATF1-D-R is: 5′-CTAGAACTAGTGGATCTTTACTTGAGTGATTAGTGAT-3′.

A CsATF1 gene knockout mutant, comprising following preparation steps: introducing the knockout vectors constructed and obtained as described above into a colletotrichum protoplast of Hevea brasiliensis, screening through a DCM medium containing chlorimuron-ethyl, and then PCR validating and sequencing validating to obtain the CsATF1 gene knockout mutant.

Application of the colletotrichum transcription factor CsATF1 and/or the protein encoded by the colletotrichum transcription factor CsATF1 in preparing a bactericidal enhancer.

Application of at least one of the colletotrichum transcription factor CsATF1, the protein encoded by the colletotrichum transcription factor CsATF1, the CsATF1 gene knockout vector and the CsATF1 gene knockout mutant in regulating sensitivity of fungi to pyrrole agents.

Compared with the prior art. the present invention has the beneficial effects that:

According to the present invention, the transcription factor CsATF1 gene is cloned, and the results of functional experiments show that the transcription factor is involved in regulating sensitivity of fungi to pyrrole agents such as fludioxonil. Therefore, all drugs that can activate expression of the transcription factor CsATF1 may improve sensitivity of fungi to fludioxonil, and serve as enhancers for pyrrole agents such as fludioxonil to improve the sterilization efficiency.

### BRIEF DESCRIPTION OF THE DRAWINGS

FIG. 1: Schematic diagram of CsATF1 gene knockout;

FIG. 2: PCR validation of a gene deletion mutant ΔCsATF1; validation primer A is CsATF1-Ou-F/CsATF1-2R, validation primer B is CsATF1-Ou-F/ILV-2R;

FIG. 3: Colony growthform(5d) of conidia of a wild-type HN08 strain and a mutant ΔCsATF1-27 strain in CM media of different fludioxonil concentrations;

FIG. 4: Schematic diagram of vector pCX62-S construction.

### DETAILED DESCRIPTION OF THE INVENTION

In order to help better understand the technical content of the present invention, specific embodiments are provided below to further explain the present invention.

### Embodiment 1 Cloning of Hevea Brasiliensis Colletotrichum CsATF1 Gene

According to the sequence of C. graminicola transcription factor bZIP gene obtained from NCBI (registered number XM008094996.1), obtain homologous sequences by comparison in a HN08 transcriptome database for hevea brasiliensis colletotrichum by a local BLAST method, design a primer pair CsATF1-F (5′-ATGGGAACTTCGCCGACCGAC-3′)/CsATF1-R(5′-TCATGAGAAACGTCGCTGGA-3′), and use cDNA and DNA of C. siamense HN08 as templates for separate amplification to obtain target bands. Sequence analysis shows that: the obtained sequence contains a complete coded open reading frame. The DNA sequence size is 1758 bp, and the cDNA sequence size is 1611 bp; the gene contains 2 introns, encodes 536 amino acids and contains 3 Aft1 structural domains and 1 BRLZ (basic leucine zipper) domain, indicating that CsATF1 is an ATF transcription factor in the bZIP transcription factor family. The gene is named CsATF1.

### Embodiment 2 Construction of CsATF1 Gene Knockout Vector and Obtainment of CsATF1 Gene Knockout Mutant

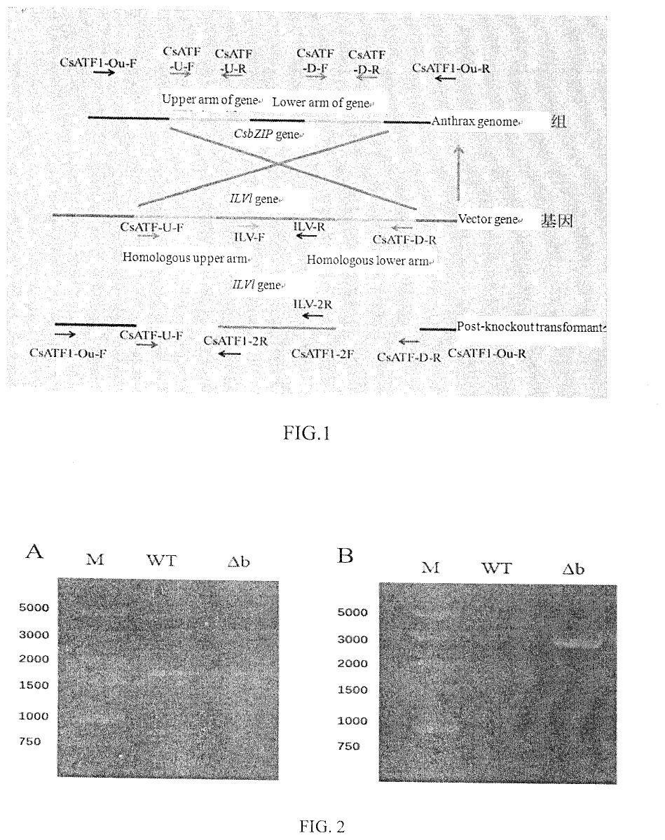

Design primer pairs CsATF1-UP-F/CsATF1-UP-R and CsATF1-D-F/CsATF1-D-R at front and rear ends of a CsATF1 gene encoded reading frame, obtain an upper arm sequence of the CsATF1 gene and a lower arm sequence behind a C terminal by PCR amplification, and link the upper arm sequence and the lower arm sequence into a vector pCX62-S by a method of homologous recombination to obtain a knockout vector pCX62-S-CsATF1. See FIG. 1 for the detailed schematic diagram. Introduce the knockout vectors constructed and obtained as described above into a C. siamense HN08 protoplast by a method of PEG-mediated protoplast transformation, and screen through the DCM medium containing chlorimuron-ethyl (100 μg/mL). A total of 3 batches are transformed, and 159 transformants are obtained.

Extract a genomic DNA sequence of 159 transformants in separate batches with PCR validation, a transformant ΔCsATF1-27 is as expected, an upstream primer CsATF1-Ou-F (5′-GAAGCAGGAGCAACATGGAA-3′) of the CsATF1 gene and an inner primer CsATF1-2R(5′-TGAGTCCAGCTATGCTGTCCG-3′) of the CsATF1 gene (FIG. 1), the mutant cannot amplify to the band, but the wild-type HN08 can amplify to a target band with a size of around 1700 bp (FIG. 2, A); the mutant can amplify to a target band with a size of about 2800 bp using an inner primer ILV-2R (5′-GTGAGAGCATGCAATTCCCGTGCAATA-3′) of a chlorimuron-ethyl resistance gene ILV and an upstream primer CsATF1-YZ-F (5′-GAAGCAGGAGCAACATGGAA-3′) of the CsATF1 gene, and this band is not amplified in the wild-type HN08 (FIG. 2, B). PCR results preliminarily showed that the CsATF1 gene in the transformant ΔCsATF1-27 had been replaced by the ILV1 gene.

Using the primer pair CsATF1-Ou-F/CsATF1-Ou-R, amplify a 4853 bp-long band, and submit the band to BGI Genomics for sequencing; sequence analysis shows that the target gene, i.e. CsATF1 gene, had been replaced by the ILV1 gene. It is confirmed that the transformant ΔCSATF1-27 is a CsATF1 gene deletion mutant.

### Embodiment 3 CsATF1 Gene Deletion Affects Sensitivity of Fungi to a Bactericide Fludioxonil

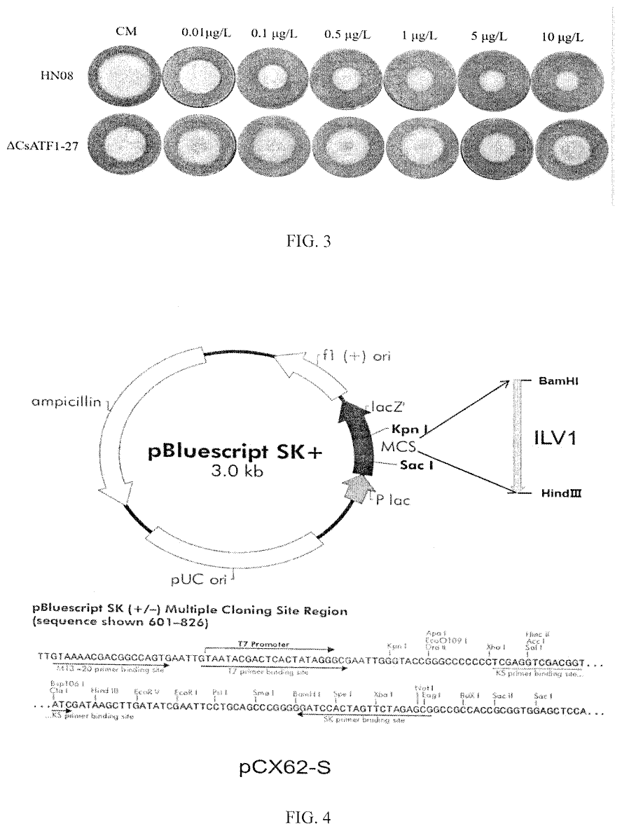

In media containing fludioxonil of different concentrations, with the increasing concentrations of fludioxonil, the strain growth rate of wild-type strains containing CsATF1 genes gradually decreases. But in the CsATF1 gene deletion mutant, the mutant has a higher growth rate than that of the wild-type strains on media with the same concentration of fludioxonil; see FIG. 3. This result indicates that the transcription factor CsATF1 can realize regulation of sensitivity of fungi to pyrrole agents such as fludioxonil, and the expression of this gene can increase sensitivity of fungi such as colletotrichum to pyrrole agents such as fludioxonil.

The above statements are only preferred embodiments of the present invention and are not intended to limit the present invention. Any modification, equivalent replacement, improvement and the like made within the spirit and principle of the present invention shall be included in the protection scope of the present invention.

What is claimed is:

1. A colletotrichum transcription factor CsATF1, comprising a nucleotide sequence shown in SEQ ID NO:1.

2. A protein encoded by the colletotrichum transcription factor CsATF1 according to claim 1, wherein the protein contains an amino acid sequence shown in SEQ ID NO:2.

3. A method for preparing a CsATF1 gene knockout vector, comprising the preparation steps of:
designing primer pairs CsATF1-UP-F/CsATF1-UP-R and CsATF1-D-F/CsATF1-D-R at front and rear ends of a CsATF1 gene encoded reading frame,
obtaining an upper arm sequence of the CsATF1 gene and a lower arm sequence behind a C terminal by PCR amplification, and
linking the upper arm sequence and the lower arm sequence into a vector pCX62-S by a method of homologous recombination to obtain the knockout vector; and the nucleotide sequence of the CsATF1 gene is shown as SEQ ID NO:1;
the nucleotide sequence of primer CsATF1-UP-F is: 5′-GTACCGGGCCCCCCCAGCTGAAGCAGGAGCAACATGGAA-3′
the nucleotide sequence of primer CSATF1-UP-R is: 5′-CGATACCGTCGACCTCGAGATGACGACGATGATGTATT-3′
the nucleotide sequence of primer CsATF1-D-F is: 5′-GCTCTCACCGCGGATCCGAGAAGTGATGCGTAATCTG-3′
the nucleotide sequence of primer CsATF1-D-R is: 5′-CTAGAACTAGTGGATCTTTACTTGAGTGATTAGTGAT-3′.

4. A method for preparing a CsATF1 gene knockout mutant, comprising the following preparation steps of:
introducing the CsATF1 gene knockout vector, constructed and obtained in claim 3 into a hevea brasiliensis colletotrichum protoplast, screening through a DCM medium containing Chlorimuron-ethyl, and then PCR validating to obtain the CsATF1 gene knockout mutant.

5. An application for preparing a bactericidal enhancer, by using the colletotrichum transcription factor CsATF1 comprising the nucleotide sequence shown in SEQ ID NO:1 and/or the protein containing an amino acid sequence shown in SEQ ID NO:2.

6. An application for regulating sensitivity of fungi to pyrrole agents, by using at least one of a colletotrichum transcription factor CsATF1 comprising a nucleotide sequence shown in SEQ ID NO: 1, a protein encoded by the colletotrichum transcription factor CsATF1 containing an amino acid sequence shown in SEQ ID NO:2, the CsATF1 gene knockout vector according to the claim 3 and a CsATF1 gene knockout mutant comprising a hevea brasiliensis colletotrichum protoplast, screening through a DCM medium containing Chlorimuron-ethyl, and then PCR validating to obtain the CsATF1 gene knockout mutant.
